(19) United States
(12) Patent Application Publication
(10) Pub. No.: US 2024/0245290 A1
(43) Pub. Date: Jul. 25, 2024

(54) SCOPE SYSTEM DISTAL END FEATURES

(71) Applicant: Cook Medical Technologies LLC, Bloomington, IN (US)

(72) Inventors: Vincent McHugo, Birdhill (IE); Liam Breen, Ballina (IE); Jonathan K. Lupton, Thomasville, NC (US); John C. Sigmon, JR., Winston-Salem, NC (US); Shaun D. Gittard, Winston-Salem, NC (US)

(73) Assignee: Cook Medical Technologies LLC, Bloomington, IN (US)

(21) Appl. No.: 18/414,931

(22) Filed: Jan. 17, 2024

Related U.S. Application Data
(60) Provisional application No. 63/480,818, filed on Jan. 20, 2023.

(51) Int. Cl. A61B 1/12

(52) U.S. Cl. CPC A61B 1/126

(57) ABSTRACT

A scope system may comprise an elongate tube having a lumen extending therethrough. The elongate tube may have a distal portion. The scope system may further comprise at least one accessory channel extending at least partially through the lumen. The accessory channel may comprise a tubular structure having an accessory lumen extending therethrough. The accessory channel may be moveable relative to the distal portion of the elongate tube and may be configured to direct fluid to an irrigation opening located at a distal end of the accessory channel.

### RELATED APPLICATIONS

The present patent document claims the benefit of the filing date under 35 U.S.C. § 119(e) of Provisional U.S. Patent Application Ser. No. 63/480,818, filed Jan. 20, 2023, which is hereby incorporated by reference.

### FIELD

The present disclosure relates to medical devices and more specifically to endoscope systems.

### BACKGROUND

The statements in this section merely provide background information related to the present disclosure and may not constitute prior art.

The traditional endoscope is a medical device used in a variety of procedures. A physician may insert the endoscope, for example, into a patient's mouth and through the patient's gastrointestinal (GI) tract. The physician may then use a variety of instruments during the procedure that are passed through an accessory channel that is located within the outer shaft of the endoscope. As the endoscopy field advances, new endoscopes are being created for specific procedures.

An endoscope system may be used as a duodenoscope. The duodenoscope is a medical device used in a variety of endoscopic procedures, including endoscopic retrograde cholangio-pancreatography (ERCP). In an ERCP, a physician inserts the duodenoscope into a patient's mouth, through the patient's gastrointestinal (GI) tract, and into the duodenum until the distal end of the duodenoscope is positioned near the papilla of Vater, a small mound-like structure that acts as the entrance from the common bile duct and pancreatic duct into the duodenum. The physician then uses a variety of tools and accessories that are passed through a lumen in the duodenoscope to access the common bile duct or pancreatic duct through the papilla of Vater.

United States patent Applications and Patents, US 2020-0163534 A1, US 2017-0251908 A1, and U.S. Pat. No. 10,582,835 B2 are hereby incorporated by reference and provide a scope system that has both a camera channel containing the camera catheter and a working channel. This working channel allows for standard diameter endoscopic instruments to be used with the scope. Further, the camera catheter may function both a duodenal scope and a Cholangioscope. The arrangement of the camera channel and working channel allows for the camera catheter to be used side by side with the endoscopic instrument up in the biliary tree for direct Cholangioscopic visualization of the instrument. However, this scope configuration presents a number of new challenges.

For example, when performing an ERCP procedure with a duodenoscope configuration, endoscopic vision may be provided by a light source and a camera for navigating and instrument use. The camera lens or the light source can become occluded by residues present with the GI tract, and so the scope is unusable. When performing Cholangioscopy, endoscopic vision is provided by a light source and a camera for navigating and instrument use. Residues or stone fragments present with the GI tract can occlude the camera lens or the light source, and so the scope is unusable. Ability to remove this contamination reliably is a benefit.

Additionally, when cannulating the bile duct in cholangioscope configuration, the physical size of the anatomy is challenging. As the scope travels in the liver direction, the inner diameter of the lumens become progressively smaller. Often the area of disease is in a region where the duct is very narrow. It is challenging to access these smaller diameter regions while providing the physician with endoscopic vision coupled with the ability to provide clinical diagnostics and therapeutics.

### SUMMARY

In one form of the present disclosure a scope system is provided. The scope system may comprise an elongate tube having a lumen extending therethrough. The elongate tube may have a distal portion. The scope system may further comprise at least one accessory channel extending at least partially through the lumen. The accessory channel may comprise a tubular structure having an accessory lumen extending therethrough. The accessory channel may be moveable relative to the distal portion of the elongate tube and may be configured to direct fluid to an irrigation opening located at a distal end of the accessory channel.

Additionally or alternatively, the accessory channel may comprise a tubular structure having an accessory lumen extending therethrough and an accessory may extend through the accessory channel. The accessory may include an irrigation lumen leading to the irrigation opening at a distal end of the accessory. The accessory may have a distal portion with a first portion of a stop feature disposed on the distal portion of the accessory. The accessory channel may extend through the distal end of the scope system. The distal end may comprise a second portion of the stop feature. The first portion of the stop feature and the second portion of the stop feature may allow for axial alignment of the distal portion of the accessory to the distal end of the scope system.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1 illustrates an example of a scope system;

FIG. 2 illustrates another view of the scope system;

FIGS. 3A-3B illustrate cross-sectional views of a distal end of the scope system, where FIG. 3A is an end-on view from the distal end;

FIGS. 4A-4B illustrate another view of the distal end of the scope system;

FIG. 5 illustrates another view of the distal end of the scope system;

FIGS. 6A-6B illustrate another view of the distal end of the scope system;

FIGS. 7A-7B illustrate another view of the distal end of the scope system;

FIG. 8 illustrates a schematic view of the distal end of the scope system;

FIG. 9 illustrates an example of a water jet of the scope system;

FIGS. 10A-10B illustrate another view of the distal end of the scope system;

FIG. 11 illustrates another cross-sectional view of the distal end of the scope system; and

FIG. 12 illustrates another view of the distal end of the scope system.

### DETAILED DESCRIPTION

The following description is merely exemplary in nature and is not intended to limit the present disclosure, application, or uses. It should be understood that throughout the drawings, corresponding reference numerals indicate like or corresponding parts and features.

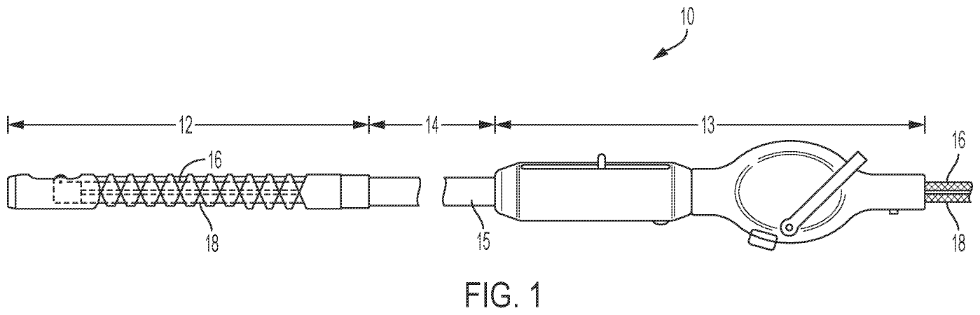

Referring to FIG. 1, an scope system 10 is provided. The scope system 10, may, for example, be an endoscope system 10. The scope system 10 may be generally shaped as an elongate tube including a distal portion 12, a central portion 14, and a proximal, or handle, portion 13. The central portion 14 may be a flexible, elongate tube with at least one lumen 15 running throughout the length of the central portion 14. The central portion 14 may connect the distal portion 12 and proximal portion 13 together. The lumen 15 of the central portion 14 may extend through the distal 12 and handle portions 13 of the scope system 10 as well. The central portion 14 may be made of a braided material such as pebax with a polytetrafluoroethylene liner to provide sufficient torqueability and pushability. Other potential materials for the central portion 14 include but are not limited to polyethylene, polypropylene, and nylon. The scope system 10 may further include two accessory channels 16, 18 each with lumens running therethrough. The accessory channels 16, 18 may be designed as individual elongated tubes that may be movable within the lumen 15 of the system 10, thus allowing longitudinal movement of the accessory channels 16, 18 with respect to the central portion 14. While this embodiment includes two accessory channels 16, 18, one or even three or more accessory channels may be used. For example, a single, larger accessory channel may be used to accommodate larger endoscopic tools. Further, in lieu of individual accessory channels 16, 18, a single elongate tube may be used with two or more lumens running through it. The accessory channels 16, 18 may range in diameter anywhere from 1 to 10 millimeters. In one exemplary embodiment, the first accessory channel 16 may be for example, 3.7 millimeters in diameter while the second accessory channel 18 may be, for example, 4.2 millimeters in diameter. The accessory channels may be, for example, circular in cross-section. Alternatively, they may be non-circular and/or irregular in cross-section. The accessory channels 16, 18 may extend proximally from or past the handle portion 13, through the lumen 15 and into the distal portion 12. Various tools, devices, and cameras may be inserted into and removed from the accessory channels 16, 18.

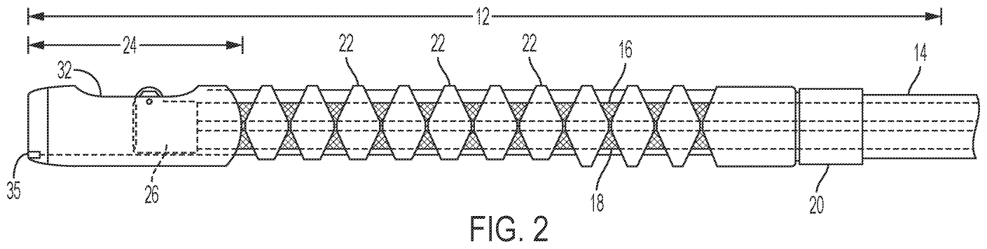

Now referring to FIG. 2, a detailed view of the distal portion 12 of the scope system 10 is shown. The scope system 10 may include a rotational bearing 20 disposed between the central portion 14 and the distal portion 12, which allows the distal portion 12 to rotate independently of the central portion 14. The distal portion 12 may have a flexible rib-like construction with multiple individual ribs 22 connected together to create an elongate tube with a lumen 15. These ribs 22 may be made of a variety of materials, such as polycarbonate, nylon, polyethylene, polypropylene, and polyoxymethylene. The accessory channels 16, 18 may travel through the ribs 22 to the distal end section 24 of the distal portion 12. The distal end section 24 may include a pivot arm 26 with first and second accessory lumens. The distal ends of the accessory channels 16, 18 may be fixedly or movably disposed within respective accessory lumens.

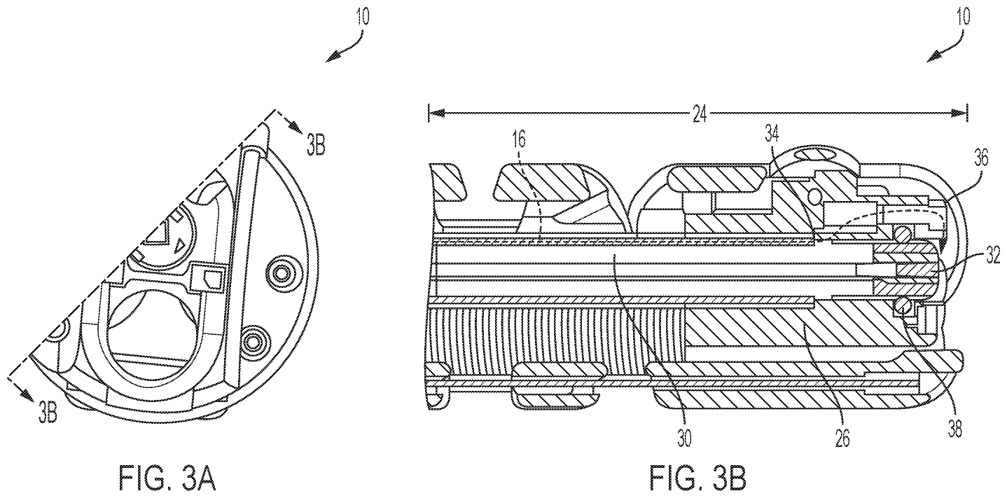

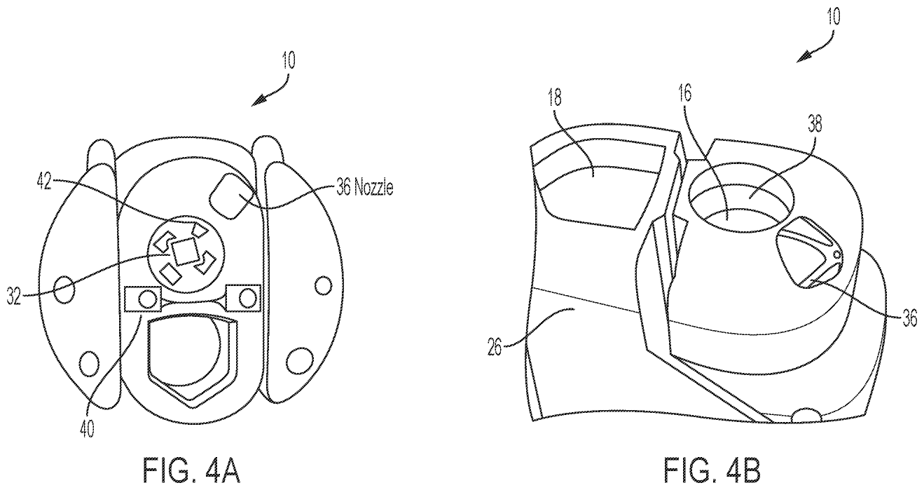

The distal end section 24 of the distal portion 12 is shown in more detail in FIGS. 3 and 4A-4B. The pivot arm 26 may be connected to the distal end section 24 via a pin. The pin may create a pivot point, around which the pivot arm 26 may rotate with respect to the distal end section 24. The pivot arm 26 may be moved between a forward-viewing position and a side-viewing position. A light 35, for example, an LED light, may be placed on the distal end section 24 to assist in navigation through a patient's GI tract. Alternatively, the light 35 may be placed at other locations on the distal end section 24. Also, multiple LED lights 35 may be used at various locations on the system 10.

FIG. 3 illustrates a close up, cross-sectional view of the distal end section 24. At least one of the accessory channels 16, 18 may be a camera channel. A camera 32 and/or camera catheter 30 may be removably disposed within the camera channel 16. Any suitable accessory, for example the camera catheter 30, may be removably disposed within either of the accessory channels 16, 18. One such accessory may be, for example, the catheter 30, for example, a camera catheter 30. The accessory may comprise an accessory tip disposed at the distal most end of the accessory. For example, in the instance of the camera catheter 30, a camera 32 may be disposed at the accessory tip of the camera catheter. The accessory tip, the tip of the camera 32, and/or the tip of the camera catheter 30 may, for example, have an atraumatic bullet shape. The camera channel 16 may further comprise a camera channel irrigation opening 34, a nozzle 36, and an O-ring 38.

The camera channel 16 may be any one of the accessory channels 16, 18 within the scope system 10. The camera channel 16 may include a lumen extending through the camera channel 16. The cameral channel 16 may be, for example, tubular in structure with an inner diameter of 3.7 millimeters. The cameral channel 16 may, for example, be ovular with a major axis of 3.3 millimeters by 2.8 millimeters. The camera channel 16 may be able to move within the scope system 10 relative to the lumen 15 and/or outer structure of the scope system 10. For example, the camera channel 16 may move relative to the distal portion 12 and/or distal end section 24 of the scope system 10. The camera channel 16 may move relative to an intermediate portion of the scope system 10, and/or more portions of the scope system 10 closer to the proximal end of the scope system 10 than the distal portion 12.

The camera catheter 30 may any structure able to be removably disposed within the camera channel 16 and able to be advanced and/or retracted past the distal end section 24. The camera catheter 30 may comprise, for example, PEBAX, nylon, fluoropolymers, FED, PTFE, and/or polyamide. The camera catheter 30 may have one or more lumens disposed within the structure of the camera catheter 30. The lumens extend the length of the camera catheter 30. The camera catheter 30 may comprise a long, cylindrical structure that extends the length of the scope system 10 from the proximal end of the scope system 10 to the distal end section 24. The camera catheter 30 may extend past the distal end section 24, for example, when the scope system 10 is in operation and the camera channel 16 and/or camera catheter 30 is extended and/or retracted.

The camera 32 may be, for example, a high resolution camera. The camera 32 may be, for example, a complementary metal-oxide semiconductor (CMOS) or charged coupled device (CCD) type camera. The camera 32 may, for example, have a resolution of at least 200×200 pixels or higher. Alternatively or additionally the camera 32 resolution may be 720, 1080p, or 4 k. Additionally or alternatively, the camera 32 may, for example, have a depth of field of 1.5 mm-100 mm, a field of view of 90°-1700°, a resolution of 200×200 to 8000×8000, and/or a frame rate or 20-60 fps. The camera 32 may be disposed at a distalmost end of the camera catheter 30, for example, at the distal tip (accessory tip) of the camera catheter.

The camera catheter 30 may be disposed within the camera channel 16 and the camera catheter 30 may have a cross section and/or diameter that is smaller in size than a cross section and/or diameter of the camera channel 16. This design may enable a flow of fluid, for example water, to run through the camera channel 16, for example between an inner wall of the camera channel 16 and an outer wall of the camera catheter 30, enabling at least a portion of the camera channel 16, for example, the open space in the camera channel 16 surrounding the camera catheter 30, to act as a fluid channel for the fluid to flow through. The fluid channel may be in fluid communication with a fluid source, for example, a fluid source external to the scope system 10. The fluid channel may be in fluid communication with the camera channel 16, the irrigation opening 34, and/or the nozzle 36. The flow of fluid may start from the fluid source, flow through a proximal end of the scope system 10, through the length of the scope system 10, though the length of the camera channel 16, for example, the through the fluid channel formed between the internal wall of the camera channel 16 and the external wall of the camera catheter 30, and to the camera channel irrigation opening 34.

The camera channel irrigation opening 34 may be any opening in the cameral channel 16. The camera channel irrigation opening 34 may, for example, may be an opening or aperture near the distal end of the camera channel 16. The camera channel irrigation opening 34 may allow fluid flowing through the camera channel 16 to exit the camera channel 16 and/or the fluid channel, for example, at the distal end of the camera channel 16. The camera channel irrigation opening 34 may be disposed at the distal most end of the camera channel 16, or, additionally or alternatively, along the wall of the camera channel 16 between the distal and proximal ends of the camera channel 16. The camera channel irrigation opening 34 may, for example, be one or more holes or apertures in the wall of the camera channel 16. Additionally or alternatively, the camera channel irrigation opening 34 may extend partially or wholly around the diameter of the camera channel 16 wall.

The camera channel irrigation opening 34 may, for example, be disposed near the nozzle 36. The nozzle 36 may be disposed at a distal end of the scope system 10, for example, near a distal end of the camera channel 16 and/or near a tip of one or more of the accessory channels 16, 18. The nozzle 36 may be bent, for example, in an “L” shape, with one end of the “L” disposed relatively parallel to the camera channel 16 and camera catheter 30, and the other end of the “L” bent substantially perpendicular to the camera channel 16 and camera catheter 30 and be disposed, for example, near a lens of the camera 32 such that fluid flowing through the nozzle 36 from the camera channel 16 is routed to an outer surface of the camera 32 lens. The camera channel irrigation opening 34 may, for example, allow for fluid to flow from the camera channel 16 and/or the fluid channel, through the camera channel irrigation opening 34, and into the nozzle 36.

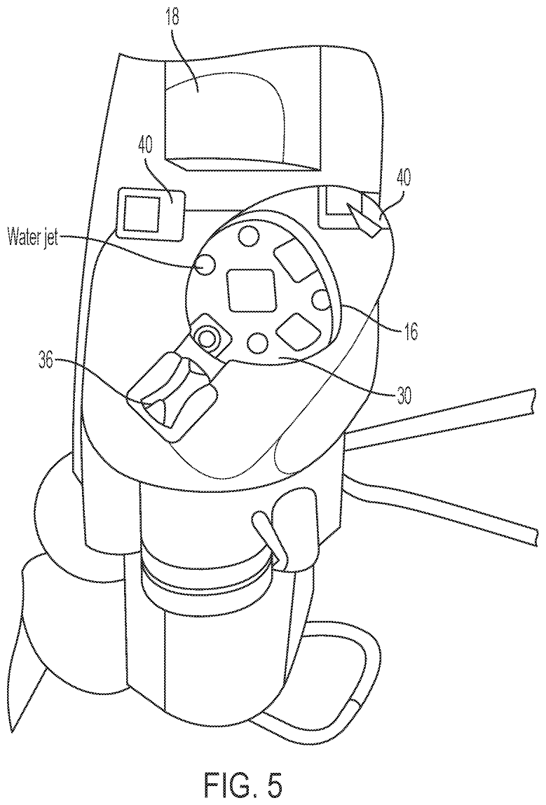

The camera channel irrigation opening 34 and nozzle 36 may be mounted on the pivot arm 26 adjacent to the camera 32, which may further allow a jet of the fluid to be flushed across a lens of the camera 32 (see FIGS. 4 and 5). Mounting the camera channel irrigation opening 34 and nozzle 36 on the pivot arm 26 may enable the jet of fluid to clean the camera 32 when the scope system 10 is in both forward view and side viewing configurations, for example, when the camera channel 16 is rotated about the pin of the pivot arm. The camera channel irrigation opening 34 and nozzle 36 may be positioned so that the jet of fluid may also clean a pivot arm light source 40 (see FIG. 4). Additionally or alternatively, the camera channel irrigation opening 34 and nozzle 36 may be positioned so that the jet of fluid cleans a camera catheter light source 42 (see FIG. 4).

The O-ring 38 may be disposed at the distal end of the camera channel 16 and may act as a fluid-tight seal between the outer wall of the camera catheter 30 and the inner wall of the camera channel 16. The O-ring 38 may be any material capable of creating such a seal. The O-ring 38 may extend, for example, around the diameter of the camera catheter 30 and may ensure that fluid in the camera channel 16 only exits through the camera channel irrigation opening 34. The O-ring 38 may be disposed near a distal end of the camera catheter 30 and/or camera channel 16. The O-ring 38 may, for example, fit into a groove disposed in the inner wall of the camera channel 16.

The fluid used may be routed through the fluid channel, for example, in the space between the camera catheter 30 and the camera channel 16. The O-ring 38 may be provided in the pivot arm 26 to create a seal with a tip of the camera catheter 30 such that the fluid is directed through the irrigation opening 34 and nozzle 36. This configuration eliminates the requirement for a dedicated water channel within the shaft of the scope system 10.

FIGS. 4A and 4B illustrate another view of the distal end section 24 of the scope system 10, the accessory channels 16, 18, the camera 32, the camera catheter 30, the nozzle 36, the pivot arm 26, the O-ring 38, the pivot arm light source 40, and the camera catheter light source 42.

There may be a plurality of light sources on the scope system 10. For example, there may be one or more pivot arm light sources 40 disposed at, for example, the distal most end of the pivot arm 26. These light sources may extend through lumens and/or accessory channels running through the scope system, with a light at the end of the pivot arm light source 40 disposed on the distal tip of the pivot arm 26. Additionally or alternatively, there may be one or more camera catheter light sources 42. The camera catheter light sources 42 may extend through lumens disposed inside of the camera catheter 30, they may extend the length of the camera catheter 30, and may comprise a light disposed at the distal end of the camera catheter 30. The light sources may, for example, be LEDs, or any other light sources that may provide light to the distal end of the scope system 10, for example, during operation of the scope system 10.

FIG. 5 illustrates another view of the distal end section 24 with the jet of water being delivered by the nozzle 36. FIG. 5 illustrates a view of the nozzle 36 delivering the jet of water to the lens of the camera 32 and the pivot arm 26.

The shape of the camera irrigation opening 34 and the outer surface of the camera catheter 30 tip may be shaped such that the surface tension of the fluid, for example, a jet of water, causes a portion of the jet of water to divert across the lens of the camera 32 to wash it. Additionally or alternatively, if the tip of the distal end section 24 is submerged in water, the jet of water may, for example, create turbulence over the camera 32 and clean the lens and/or clear debris from the view the camera 32.

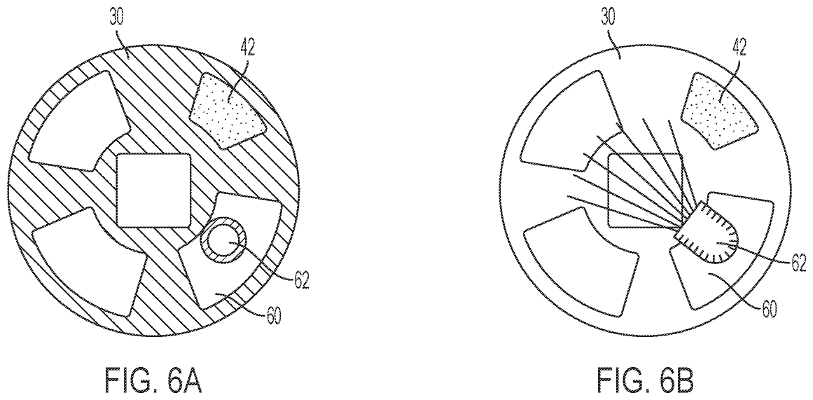

FIGS. 6A and 6B illustrate another view of the tip of the distal end section 24, where the camera catheter 30 comprises a camera catheter irrigation lumen 60 and a fluid diversion feature 62. FIG. 6A illustrates an axial cross-sectional view of the tip of the cameral catheter 30. The irrigation lumen 60 may be disposed inside of the camera catheter 30, extend the length of the camera catheter 30, and may allow for a fluid to flow from a source, through the camera catheter 30, and be delivered to the distal tip of the camera catheter 30. The camera catheter 30 may have multiple lumens disposed within the camera catheter 30, wherein one or more of the lumens act as irrigation lumens 60. A cross-section of the irrigation lumen 60 may, for example, extend around a portion of the diameter of the camera catheter 30. For example, the camera 32 may be disposed in a lumen extending through the center of the camera catheter 30, while the irrigation lumen 60 is disposed between the camera lumen and the outer wall of the camera catheter 30, the cross section of the irrigation lumen 60, for example, takes up roughly a quarter of the cross section of the camera catheter 30 between the central camera lumen and the outer wall of the camera catheter 30.

FIG. 6B illustrates the camera catheter irrigation lumen 60 and a fluid diversion feature 62, with the fluid diversion feature 62 spraying fluid across the lens of the camera 32. The camera catheter irrigation lumen 60 and/or a fluid diversion feature 62 may be circular in cross-section. The fluid division feature 62 may form an “L” shape, for example, similar to the shape of the nozzle 36, to divert fluid to the camera 32. The fluid diversion feature 62 may be attached to the camera catheter irrigation lumen 60 at the side of the “L” shaped cannula, with one end of the fluid diversion feature 62 in fluid communication with and extending parallel to the irrigation lumen 60, and the other end of the fluid diversion feature bent and extending substantially parallel to an outer surface of the lens of the camera 32. The scope system 10 may, for example, comprise both the fluid channel and nozzle 36, allowing for fluid to flow through the camera channel 16 over the camera 32 lens, as well as the irrigation lumen 60 and fluid diversion feature 62, allowing fluid to flow through the camera catheter 30 and over the camera 32 lens. Additionally or alternatively, the scope system 10 may comprise only one or the other.

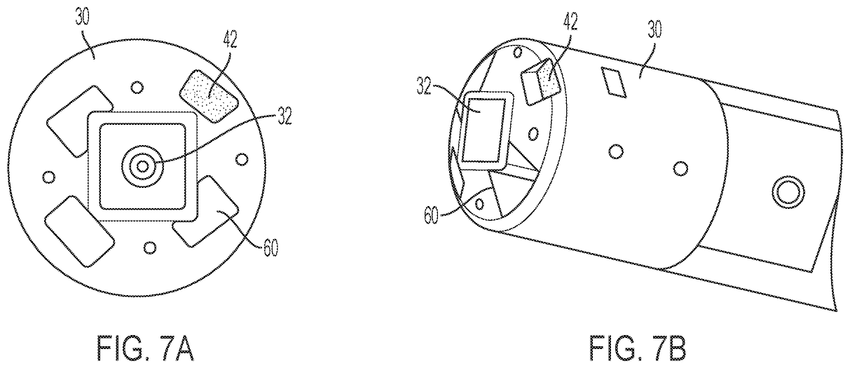

FIGS. 7A and 7B illustrate yet another view of the tip of the camera catheter 30, the irrigation lumen 60, the camera catheter light source 42, and the camera 32.

The tip of the camera catheter 30 may be a separate, injection molded component with a plurality of openings and/or lumens. For example, the tip of the camera catheter 30 may have nine lumens. The plurality of lumens may include, for example, four-way steering, two lumens for suction, a lumen for flushing, a wire guide lumen, LED Lighting, and/or a camera.

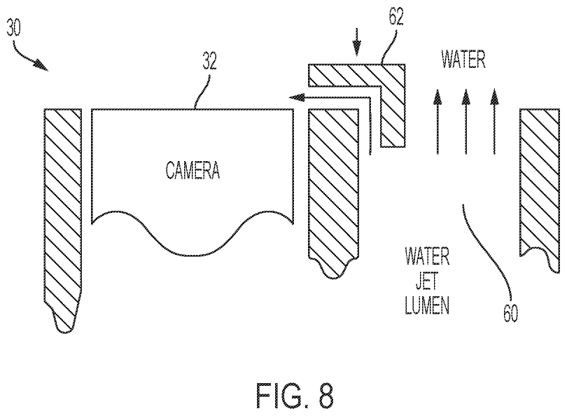

FIG. 8 illustrates a longitudinal cross-sectional view of the tip of the camera catheter 30, the camera 32, the camera catheter irrigation lumen 60, and the fluid diversion feature 62. The fluid diversion feature 62 may, for example, allow for fluid flowing from the irrigation lumen 60 to be diverted to flow across the lens of the camera 32 and/or flow past the fluid diversion feature 62 and flow outward from the irrigation lumen 60, flowing substantially parallel to the irrigation lumen 60. This design may allow for the irrigation lumen 60 and the fluid flowing through the lumen 60 to be used to clean the lens of the camera 32 and/or to flow out from the tip of the camera catheter 30 and clear the path of debris that may be in the way of the camera 30. These two flows of fluid may happen simultaneously, together or separately, one at a time during different parts of operation of the scope system 10. The nozzle 36 and irrigation opening 34 disposed on the distal end of the camera channel 34 and/or pivot arm 26 may also allow for a similar dual direction jet of fluid. Additionally or alternatively, in the scope system 10, the tip of the camera catheter 30 and the fluid diversion feature 62 may be designed to divert a portion of the jet of water across the camera lens to wash it. If the tip is submerged in water, turbulence will be created over the camera to clear the view.

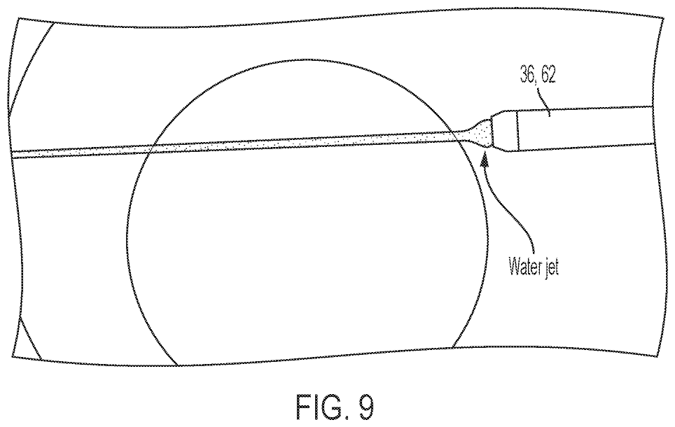

FIG. 9 illustrates an example of a jet of fluid, for example, water diverted by the nozzle 36 and/or the fluid diversion feature 62.

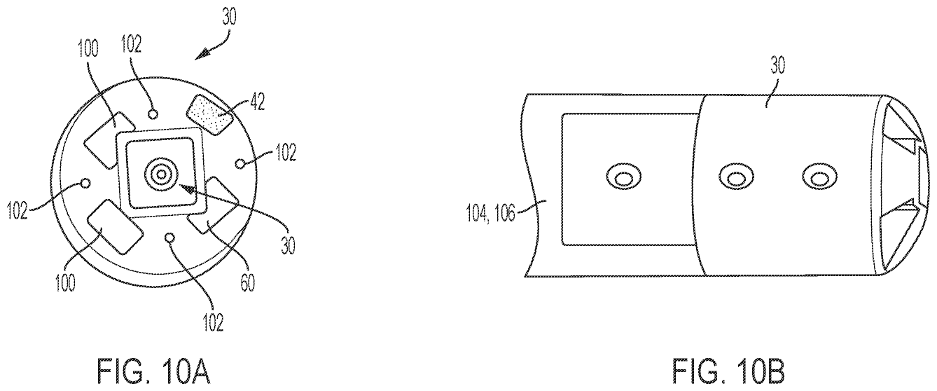

FIGS. 10A and 10B illustrate another view of the tip of the camera catheter 30. FIG. 10A shows a front view of the tip of the camera catheter 30 and the plurality of lumens. The plurality of lumens may include, for example, a lumen for the camera catheter light source 42, the camera catheter irrigation lumen 60, a suction lumen 100, and lumen through which run deflection wires 102. The lumens may extend axially thought the length of the camera catheter 30 and the devices and/or accessories disposed in the lumens (lights, wires, cameras, components needed for irrigation and/or suction, etc.) may be extended and/or retracted through the lumen along the length of the scope system 10 and past the distal end section.

FIG. 10B shows a side view of the camera catheter 30 and a first portion 104 of a stop feature 106. The distal portion of the camera catheter 30 may be designed to comprise the first portion 104 of the stop feature 106. The stop feature 106 may, for example, ensure precise axial, radial, and/or and longitudinal alignment of the camera 32 and/or the camera catheter light source 42 disposed in the tip of the camera catheter 30 with the nozzle 36 when, for example, the camera catheter 30 is docked and/or fully seated in the distal pivot arm 26 of the scope system 10.

The first portion 104 of the stop feature 106 may, for example, comprise a haptic, positive, geometric feature that protrudes out from the outer wall of the camera catheter 30. For example, the camera catheter 30 may have a relatively smaller diameter at and/or along a portion of the camera catheter 30 closer toward the proximal end of the scope system 10, and the first portion 104 of the stop feature 106 may comprise an increase in diameter of the camera catheter 30 at a portion of the camera catheter 30 closer toward and/or near the distal end of the camera catheter 30. The first portion 104 may be as simply a uniform increase in diameter and/or geometry of the camera catheter 30 near the distal end.

Additionally or alternatively, the first portion 104 may comprise more complex geometry such that the camera catheter is both aligned longitudinally, radially, and/or axially when docked with the pivot arm 26. For example, the first portion 104 may comprise symmetrical, asymmetric, or irregular geometry, for example, protrusions shaped, for example, like chevrons. The first portion 104 may, for example, comprise a chevron-shaped protrusion with the point or tip of the chevron pointed towards the proximal end of the scope system 10. The first portion 104 may comprise protrusions, for example, one or more chevron protrusions disposed on either side on the camera catheter 30 opposite from one another, such that the first portion 104 will cause the tip of the camera catheter 30 to rotated until a precise axial alignment is achieved and the first portion 104 can be fully docked or seated flush against matching geometry disposed within the tip of the pivot arm 26.

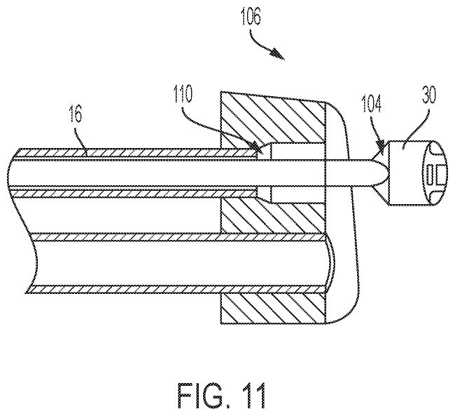

FIG. 11 illustrates another cross-sectional view of the distal end section 24, with the camera catheter 30, the tip of the pivot arm 26, and a second portion 110 of the stop feature 106. The second portion 110 of the stop feature 106 may be disposed in the distal end section 24 of the scope system 10, for example, at the distal end of the pivot arm 26 and/or at the distal end of the camera channel 16. The second portion 110, for example, may comprise specific geometry of the inner wall of the camera channel 16 where the tip of the camera catheter 30 sits when fully seated in the pivot arm 26. For example, the second portion 110 may be a negative match of positive geometric protrusion of the first portion 104. For example, when the camera catheter 30 is fully retracted and/or docked within the distal end section 24 and/or the pivot arm 26, the outer surface of the first portion 104 may contact and/or sit flush against the surface of the second portion 110.

The stop feature 106 may be a positive stop feature. The stop feature 106 may comprise the first portion 104 of the stop feature 106 disposed on or near the distal end of the camera catheter 30 and the second portion 110 disposed on the pivot arm 26 of the scope system 10. The second portion 110 may engage with the first portion 104 on the camera catheter 30 when the camera catheter is docked, or fully seated in the pivot arm 26, for example, when reverting the scope system 10 from cholangioscope to duodenoscope. The stop feature 106 may ensure that the camera 32 is aligned precisely with the nozzle 36. Additionally or alternatively, the shape of the jet of the fluid from the nozzle 36 may also be altered or multiple jets provided to reduce the need for precise alignment of the camera catheter 30 to the pivot arm 26 and nozzle 36.

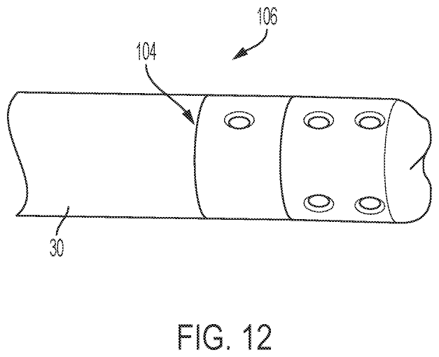

FIG. 12 illustrates yet another view of the camera catheter 30 and first portion 104 of the stop feature 106.

Each component may include additional, different, or fewer components. For example, the accessory channels 18, 16, camera catheter 30, and/or any other components may comprise fewer or additional channels, lumens, and/or accessories.

The system 10 may be implemented with additional, different, or fewer components. For example, the scope system 10 may comprise additional features, accessory or control components, channels, and/or lumens.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed. Unless otherwise indicated or the context suggests otherwise, as used herein, “a” or “an” means “at least one” or “one or more.”

While various embodiments have been described, it will be apparent to those of ordinary skill in the art that many more embodiments and implementations are possible. Accordingly, the embodiments described herein are examples, not the only possible embodiments and implementations.

The scope system 10 described herein may be used for a variety of medical procedures. While the embodiments described herein are shown in reference to the endoscopy field and endoscopic retrograde cholangiopancreatography procedures, the embodiments may be used in a variety of other medical procedures including endoscopic submucosal dissection and any other endoscopic procedure that would benefit by having multiple instruments at a time and/or the ability to see things from both the forward-viewing and side-viewing perspectives.

The description of the disclosure is merely exemplary in nature and, thus, variations that do not depart from the substance of the disclosure are intended to be within the scope of the disclosure. Such variations are not to be regarded as a departure from the spirit and scope of the disclosure.

We claim:

1. A scope system, comprising:
an elongate tube having a lumen extending therethrough, the elongate tube having a distal portion; and
at least one accessory channel extending at least partially through the lumen, the accessory channel comprising a tubular structure having an accessory lumen extending therethrough, wherein the accessory channel is moveable relative to the distal portion of the elongate tube,
wherein the accessory channel is configured to direct fluid to an irrigation opening located at a distal end of the accessory channel.

2. The scope system of claim 1, wherein a camera is located at the distal end of the accessory channel, and wherein the irrigation opening is shaped and orient to direct at least a portion of fluid as a jet towards a surface of the camera for cleaning a surface of the camera.

3. The scope system of claim 2, wherein the camera is comprised by a camera catheter that extends through the accessory lumen, and wherein the accessory channel for directing the fluid is outside the camera catheter.

4. The scope system of claim 3, wherein an O-ring surrounds the camera catheter for preventing fluid from flowing distally out of the accessory lumen.

5. The scope system of claim 2, wherein at least one light source is located at a distal end of the accessory channel, and wherein the irrigation opening directs at least a portion of the fluid jet towards the at least one light source for cleaning the light source.

6. The scope system of claim 1, further comprising a fluid channel located at the distal end of the accessory channel, the fluid channel configured to direct a fluid to the irrigation opening.

7. The scope system of claim 5, wherein a fluid channel is in fluid communication with the accessory lumen such that fluid flows from the accessory channel to the fluid channel at the distal end of the accessory channel.

8. The scope system of claim 1, wherein the irrigation opening includes at least two outlets such that a resulting jet of fluid includes at least two directions.

9. The scope system of claim 1, wherein the irrigation opening includes a nozzle configured to divert at least a portion of a jet of fluid across a surface of an accessory.

10. A scope system, comprising:
an elongate tube having a lumen extending therethrough, the elongate tube having a distal portion;
an accessory channel extending at least partially through the lumen, the accessory channel comprising a tubular structure having an accessory lumen extending therethrough; and
an accessory extending through the accessory channel,
wherein the accessory includes an irrigation lumen leading to an irrigation opening at a distal end of the accessory.

11. The scope system of claim 10, wherein the irrigation opening includes a fluid diversion feature to divert at least a portion of a jet of fluid across a surface of the accessory.

12. The scope system of claim 11, wherein the surface of the accessory is a camera lens surface.

13. The scope system of claim 11, the accessory comprises a first portion of a stop feature and the accessory channel comprises a second portion of the stop feature, wherein the first portion of the stop feature and the second portion of the stop feature allow for axial alignment of a tip of the accessory to the distal portion of the scope system.

14. A scope system comprising:
an elongate tube having a lumen extending therethrough, the elongate tube having a distal portion;
an accessory channel extending at least partially through the lumen, the accessory channel comprising a tubular structure having an accessory lumen extending therethrough;
an accessory extending through the accessory lumen, the accessory having a distal portion;
a first portion of a stop feature disposed on the distal portion of the accessory; and
a distal end of the scope system, the accessory channel extending through the distal end, the distal end comprising a second portion of the stop feature,
wherein the first portion of the stop feature and the second portion of the stop feature allow for axial alignment of the distal portion of the accessory to the distal end of the scope system.

15. The scope system of claim 14, wherein the first portion of the stop feature sits flush against the second portion of the stop feature when the accessory is fully docked in the distal end of the scope system.

16. The scope system of claim 14 wherein the first portion of the stop feature and the second portion of the stop feature are shaped such that the first portion of the stop feature and the second portion of the stop feature allow for axial alignment and radial alignment of the accessory to the scope system.

17. The scope system of claim 16, wherein the first portion of the stop feature and the second portion of the stop feature comprise alignment chevrons.

18. The scope system of claim 14 further comprising an irrigation opening disposed at a distal end of the accessory channel, wherein the first portion of the stop feature and the second portion of the stop feature align the accessory with a nozzle.

19. The scope system of claim 14, wherein the accessory comprises an atraumatic bullet shape tip.

20. The scope system of claim 14, wherein the first portion of the stop feature and the second portion of the stop feature allow for axial alignment of the accessory, a camera disposed on the accessory, and an accessory component disposed on the distal end of the scope system.
